(19) United States
(12) Patent Application Publication
(10) Pub. No.: US 2012/0164100 A1
(43) Pub. Date: Jun. 28, 2012

(54) TEMPERATURE SENSITIVE HYDROGEL AND BLOCK COPOLYMERS

(76) Inventors: Ren-Ke Li, Toronto (CA); Faquan Zeng, Toronto (CA)

(21) Appl. No.: 13/287,808

(22) Filed: Nov. 2, 2011

Related U.S. Application Data
(60) Provisional application No. 61/409,261, filed on Nov. 2, 2010.

(51) Int. Cl. A61K 35/28; C08G 63/91; A61K 38/20; A61K 38/19; A61P 9/10; C08G 63/00; A61K 38/18

(52) U.S. Cl. 424/85.2; 528/271; 525/437; 525/54.1; 514/8.1; 514/7.9; 424/93.7

(57) ABSTRACT

The present disclosure provides temperature sensitive hydrogels and block copolymers, processes for the production thereof, and therapeutic and research compositions employing these copolymers.

### CROSS-REFERENCE TO RELATED APPLICATIONS

This application claims priority to U.S. Provisional Patent Application No. 61/409,261 filed Nov. 2, 2010, and Canadian Patent Application No. 2,719,855 filed Nov. 2, 2010, both entitled “Temperature Sensitive Hydrogel and Block Copolymers.” The entire contents of the foregoing application are hereby incorporated by reference.

### FIELD OF THE INVENTION

The present disclosure relates to a temperature sensitive hydrogel and block copolymers, processes for the production thereof, conjugates of the copolymers, therapeutic and research compositions including these hydrogels and block copolymers and their uses.

### BACKGROUND OF THE INVENTION

Recently, the advance of biomaterials for myocardial tissue engineering includes in situ polymer gels containing such materials as injectable fibrin glue, matrigel, collagen, alginate gels and self-assembling peptides. These in situ polymer scaffolds (injectable extracellular matrix, iECM), by themselves or in combination with cells or biological molecules, have proven to more precisely control the myocardial microenvironment, which enhances cell transplant survival, reduces infarct expansion, and induces neovasculature formation in ischemic myocardium as well as the sustained release for delivery of growth factors and cells.

### BRIEF SUMMARY OF THE INVENTION

The present disclosure relates to a biodegradable, body temperature-sensitive hydrogel and block copolymers, which optionally act as a delivery matrix for the delivery of therapeutic compounds, e.g., vascular growth agents, and/or as a matrix or scaffold for cells. Without wishing to be bound by any particular theory it is believed that, in some embodiments, the unique honeycomb structure and pore size of the polymers of the invention are particularly advantageous for repair and/or new cell growth. The disclosure also includes a process for the preparation of such hydrogels and block copolymers.

Accordingly, the present disclosure includes a block copolymer including an A block and a B block, wherein the block copolymer has the formula:

A-B;

A-B-A; or

B-A-B;

the A block is a poly(δ-valerolactone), poly(ε-caprolactone), poly(lactide), poly(α-hydroxy acid), poly(glycolide), polyanhydride, polyester, polyorthoester, polyetherester, polyesteramide, polycarbonate, polycyanoacrylate, polyurethane, polyacrylate, or a co-polymer thereof, all of which are optionally substituted; wherein the B block is an optionally substituted polyethylene glycol or optionally substituted polypropylene glycol; wherein the A block has a number average molecular weight between 500 and 30,000 and the B block has a number average molecular weight between 500 and 10,000; wherein the optional substituents are selected from halo, OH, (C₁₋₆)-alkyl and fluoro-substituted (C₁₋₆)-alkyl; and wherein the block copolymer forms a hydrogel at a temperature of above about 30° C.

In another embodiment, the A block has a number average molecular weight between 500 and 10,000, or 1,000 and 5,000, or 1,000 and 3,000, or about 1,750. In another embodiment, the B block has a number average molecular weight of between 1,000 and 8,000, optionally between 1,500 and 8,000, or 1,500 and 5,000.

In another embodiment, the block copolymer has the formula: A-B-A.

In a further embodiment, the A block comprises a poly(δ-valerolactone), poly(ε-caprolactone), poly(lactide), poly(α-hydroxy acid), poly(glycolide) or a copolymer thereof, optionally poly(δ-valerolactone) or poly(ε-caprolactone) or copolymers thereof, or poly(δ-valerolactone).

In an embodiment of the disclosure, the B block comprises polyethylene glycol.

In another embodiment of the disclosure, the block copolymer comprises

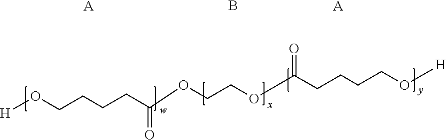

wherein the integers w, x and y represent the number of repeating units to obtain a block copolymer wherein the A block has a number average molecular weight between 500 and 30,000 and the B block has a number average molecular weight between 500 and 10,000.

In another embodiment, the molecular weight ratio of A to B may be equal to or greater than about 1.00, or about 1.05, or about 1.10, or about 1.15. The molecular weight ratio of A to B may also be equal to or less than about 1.35, or about 1.30, or about 1.25.

The present disclosure also includes a process for the preparation of a block copolymer including at least one A block and at least one B block having the formula A-B, A-B-A, B-A-B, the process including reacting (i) an optionally substituted polyethylene glycol or polypropylene oxide including the B block; with, (ii) monomeric units of the A block, the monomeric units including δ-valerolactone, ε-caprolactone, lactide, an α-hydroxy acid, glycolic acid, an anhydride, an ester, an orthoester, an etherester, an esteramide, a carbonate, a cyanoacrylate, a urethane, an acrylate, or a mixture thereof, all of which are optionally substituted, wherein the optional substituents are selected from halo, OH, (C₁₋₆)-alkyl and fluoro-substituted (C₁₋₆)-alkyl; in the presence of an acid catalyst having a pKa of less than −12, and wherein the process is optionally performed at a temperature between −10° C. and 35° C.

In some embodiments, the monomeric units of the A block are provided at a molar ratio to the B block such that the molecular weight ratio of A to B is between about 1.05 and about 1.35.

In another embodiment, the number average molecular weight of the A block is controlled by the molar ratio of the monomeric units of the A block to the B block during the process.

In another embodiment of the process, the A block has a number average molecular weight between 500 and 30,000 and the B block has a number average molecular weight between 500 and 10,000.

In another embodiment, the A block has a number average molecular weight between 500 and 10,000, or 1,000 and 5,000, or 1,000 and 3,000, or about 1,750. In another embodiment, the B block has a number average molecular weight of between 1,000 and 8,000, optionally between 1,500 and 8,000, or 1,500 and 5,000.

In another embodiment of the process, the block copolymer has the formula: A-B-A.

In another embodiment of the process, the monomeric units of the A block comprise δ-valerolactone, ε-caprolactone, lactide, an α-hydroxy acid, glycolide or a copolymer thereof, optionally δ-valerolactone or ε-caprolactone or a copolymer thereof, or δ-valerolactone.

In another embodiment of the process, the B block comprises polyethylene glycol.

In a further embodiment of the process, the acid catalyst is a sulfonic acid, optionally trifluoromethanesulfonic acid or fluorosulfonic acid. In one embodiment, the sulfonic acid is trifluoromethanesulfonic acid.

In another embodiment of the disclosure, the block copolymer including at least one A block and at least one B block, wherein the block copolymer has the formula: A-B; A-B-A; or B-A-B; in which the block copolymer is produced by the process of the disclosure. In another embodiment, the block copolymer produced by the process comprises

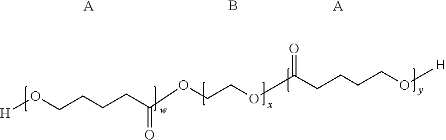

wherein the integers w, x and y represent the number of repeating units to obtain a block copolymer wherein the A block has a number average molecular weight between 500 and 30,000 and the B block has a number average molecular weight between 500 and 10,000.

The present disclosure also includes a pharmaceutical conjugate including a block copolymer as defined in the disclosure and a therapeutic compound e.g., as a vascular growth agent. In some embodiments, the therapeutic compound is a biologic, optionally stem cell factor (SCF) or vascular endothelial growth factor (VEGF), which are both vascular growth agents.

The present disclosure also includes a method for the treatment of cardiac abnormality and/or vascular abnormality in a patient in need thereof including administering a therapeutically effective amount of a pharmaceutical conjugate as defined in the disclosure to the site of the cardiovascular defect. In an embodiment, the cardiac abnormality is myocardial infarction and the vascular abnormality is a vascular aneurysm.

The present disclosure also includes a hydrogel including a block copolymer as defined in the disclosure, cells, and a therapeutic compound. In another embodiment, the cells are transplanted autologous, homologus (allogenic) or xenogenic cells and the compound is an immunosuppressant.

The present disclosure also includes a method for preventing rejection and/or prolonging the survival of transplanted autologous, homologus (allogenic) or xenogenic cells in a patient in need thereof including administering a hydrogel as defined in the disclosure.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The invention will now be described in greater detail with reference to the following drawings in which:

FIG. 1 is a photomicrograph illustrating the distribution of rat bone marrow stromal cells in a block copolymer gelled matrix in an embodiment of the disclosure;

FIG. 2 is an ¹H NMR spectrum of a block copolymer in an embodiment of the present disclosure;

FIG. 3 illustrates the characterization of a vascular endothelial growth factor conjugated to a block copolymer in an embodiment of the disclosure;

FIG. 4 is a graph illustrating the gelling temperature versus the concentration of a block polymer in an embodiment of the disclosure;

FIG. 5 shows a photograph of the gelling of a block copolymer in an embodiment of the disclosure;

FIG. 6 shows photographs heart slices after myocardial infarction;

FIG. 7 is a graph illustrating the scar area (%) after myocardial infarction;

FIG. 8 is a graph illustrating the ejection fraction after myocardial infarction; and

FIG. 9 is a graph illustrating the survival rate of implanted cells after myocardial infarction in 5 weeks.

### DETAILED DESCRIPTION OF THE INVENTION

### (I) Definitions

The term “C₁₋₆alkyl” as used herein means straight and/or branched chain, saturated alkyl radicals containing from one to six carbon atoms and includes methyl, ethyl, propyl, isopropyl, n-butyl, s-butyl, isobutyl, t-butyl and the like.

The term “halo” as used herein means halogen and includes chloro, fluoro, bromo and iodo.

The term “fluoro-substituted C₁₋₆alkyl” as used herein that at least one (including all) of the hydrogens on the referenced group is replaced with fluorine.

The term “block copolymer” as used herein refers to a polymer built of linearly linked polymeric units, prepared by the polymerization of a plurality of different monomer units in each block. The block copolymer is of the formula A-B, A-B-A, or B-A-B in which A and B represent different polymeric blocks built from repeating monomeric subunits. For example, a polyethylene glycol polymer represents one example of a B block polymer built from repeating ethylene glycol monomeric units, while poly(δ-valerolactone) represents one example of an A block polymer built from repeating δ-valerolactone monomeric units (through cationic lactone ring-opening polymerization), and as such the block copolymer for this example would be PVL-PEG.

The terms “polyethylene glycol” or “polypropylene glycol” as used herein means a polymer built from repeating ethylene glycol or propylene glycol monomeric units, respectively. Polyethylene glycol is formed of repeating units including

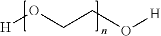

while polypropylene glycol is formed of repeating units including

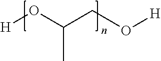

The term “temperature sensitive” hydrogel as used herein refers to a block copolymer of the present disclosure and forms, to various degrees, a jelly-like or gelled product when heated to a particular temperature, for example body temperature (37° C.), or a temperature higher than 30° C. The block copolymer is preferably a liquid at room temperature and soluble in water, but upon reaching a particular temperature, forms a hydrogel when mixed with water such that water is a dispersion medium forming the hydrogel.

The term “reverse thermal gelation temperature” as used herein is defined as meaning the temperature below which a block copolymer of the disclosure is soluble in water and above which the block copolymer solution forms a semi-solid, for example, a gel, emulsion, dispersion or suspension.

### (II) Block Copolymers of the Disclosure

The present disclosure relates to block copolymers including at least one A block which comprises hydrophobic, biodegradable, and non-swellable domains and at least one B block which comprises hydrophilic and swellable soft domains, and have the formula A-B, A-B-A or B-A-B. In one embodiment, the block copolymer is a di-block copolymer or a triblock copolymer. In one embodiment, the block copolymers are thermoplastic and biodegradable hydrogel copolymers which are liquid and dissolve in water or buffer solution at room temperature and form a hydrogel at certain temperatures, preferably at a temperature above 30° C. The block copolymers of the disclosure are biodegradable such that the copolymer erodes or degrades in vivo to form smaller non-toxic compounds.

In another embodiment, the block copolymers are thermo-sensitive hydrogels comprise amphiphilic block copolymers (including hydrophilic and hydrophobic blocks), wherein the hydrogels exhibit temperature-responsive gelation/de-gelation in addition to the reverse thermal gelation properties. In one embodiment, the block copolymer of the disclosure forms a gel by aggregation in a solution when heated to a certain temperature, and also disassociates (de-aggregates) in solution when removed from that certain temperature environment. In another embodiment, the block copolymers of the disclosure, when dissolved in solution, possess reverse thermal gelation properties in that the solution forms a gel when heated to a certain temperature, whereas typically, polymers usually lose viscosity upon heating.

In one embodiment, the temperature at which the block copolymer of the disclosure forms a hydrogel and/or aggregates is preferably at a temperature equal to or greater than about 28° C., or about 30° C., or about 32° C., or about 34° C., or about 36° C., or most preferably about 37° C. The gelation temperature is preferably greater than about room temperature and less than or equal to about body temperature. In one embodiment, the gelation temperature is preferably greater than about 22° C. and less than about 37° C.

In one embodiment, the block copolymers are easily administered to patients in need of treatment, such as syringe or catheter injection. When the block copolymers are below the gelation temperature, they may be soluble in solution for easy application. For example, block copolymers conjugated to a therapeutic compound, or containing cells and therapeutic compounds may be more easily applied at a temperature below the gelation temperature. These block copolymers are also easily processed using infusion methods or solvent casting methods because there is no chemical crosslinkage of the block copolymers. In one embodiment, the gelling of a solution of a block copolymer of the disclosure is a physical aggregation which results in the gelling of the solution, and does not involve chemical changes to the polymer (for example, chemical crosslinking).

In another embodiment, the copolymers are easily degraded into small and nontoxic molecules by simple intra-molecular ester hydrolysis or enzyme hydrolysis in order to be easily excreted through the kidney. In another embodiment, the formation of the hydrogel is reversible by heating. In one embodiment, a solution of a block copolymer of the present disclosure preferably forms a hydrogel and/or aggregates at a temperature equal to or greater than about 28° C., or about 30° C., or about 32° C., or about 34° C., or about 35° C., or about 36° C., or about 37° C.

In another embodiment, a solution of a block copolymer of the disclosure preferably returns to a solution (liquid state) when cooled to a temperature less than about 37° C., or about 35° C., or about 34° C., or about 32° C., or about 30° C. In another embodiment, a solution of a block copolymer of the disclosure preferably becomes a solution when heated to a temperature of greater than about 45° C., or about 40° C., or about 39° C. The temperature range at which the block copolymers of the disclosure form a hydrogel is preferably between about room temperature and about 45° C., or about 28° C. and about 45° C., or about 30° C. and about 45° C., or about 32° C. and about 45° C. The temperatures at which the block copolymers for a hydrogel may vary between any temperature disclosed herein at which the copolymer form a hydrogel.

Accordingly, in one embodiment the present disclosure includes a block copolymer including at least A block and at least one B block, wherein the block copolymer has the formula:

A-B;

A-B-A; or

B-A-B;

the A block is a poly(δ-valerolactone), poly(ε-caprolactone), poly(lactide), poly(α-hydroxy acid), poly(glycolide), polyanhydride, polyester, polyorthoester, polyetherester, polyesteramide, polycarbonate, polycyanoacrylate, polyurethane, polyacrylate, or a co-polymer thereof, all of which are optionally substituted; wherein the B block is an optionally substituted polyethylene glycol or optionally substituted polypropylene glycol; wherein the optional substituents are selected from halo, OH, (C₁₋₆)-alkyl and fluoro-substituted (C₁₋₆)-alkyl; and wherein the block copolymer forms a hydrogel at a temperature of above about 30° C.

In one embodiment, it will be understood by a person skilled in the art that the determination of the desired polymer degradation rate, the reverse thermal gelation temperature etc., is based upon the molecular weight of the A block polymers.

In another embodiment, the determination of the desired polymer degradation rate, the reverse thermal gelation temperature etc., is based upon the molecular weight ratio of the A block polymers to the B block polymers. The molecular weight ratio of A to B may be equal to or greater than about 1.00, or about 1.05, or about 1.10, or about 1.15, or about 1.2. The molecular weight ratio of A to B may also be equal to or less than about 1.35, or about 1.30, or about 1.25, or about 1.2. The molecular weight ratio may also range between any of these values, for example about 1.00 and about 1.25, or about 1.15 and about 1.35.

In another embodiment, to increase the rate at which the a block copolymer of the disclosure is solubilized in an aqueous solution (such as water or a phosphate buffer), the solution is heated to a temperature of greater than about 40° C., or 50° C., or optionally 60° C., and then cooled to a temperature below room temperature, or below about 20° C., or optionally below 10° C.

In another embodiment, the present disclosure includes a block copolymer including at least A block and at least one B block, wherein the block copolymer has the formula:

A-B;

A-B-A; or

B-A-B;

wherein the A block is a poly(δ-valerolactone), poly(ε-caprolactone), poly(lactide), poly(α-hydroxy acid), poly(glycolide), polyanhydride, polyester, polyorthoester, polyetherester, polyesteramide, polycarbonate, polycyanoacrylate, polyurethane, polyacrylate, or a co-polymer thereof, all of which are optionally substituted; wherein the B block is an optionally substituted polyethylene glycol or optionally substituted polypropylene glycol; wherein the A block has a number average molecular weight between 500 and 30,000 and the B block has a number average molecular weight between 500 and 10,000; wherein the optional substituents are selected from halo, OH, (C₁₋₆)-alkyl and fluoro-substituted (C₁₋₆)-alkyl; and wherein the block copolymer forms a hydrogel at a temperature of above about 30° C.

In another embodiment, the A block has a number average molecular weight between 500 and 10,000, or 1,000 and 5,000, or 1,000 and 3,000, or about 1,750. In another embodiment, the B block has a number average molecular weight of between 1,000 and 8,000, optionally between 1,500 and 8,000, or 1,500 and 5,000.

In another embodiment, the block copolymer has the formula: A-B-A.

In a further embodiment, the A block comprises a poly(δ-valerolactone), poly(ε-caprolactone), poly(lactide), poly(α-hydroxy acid), poly(glycolide) or a copolymer thereof, optionally poly(δ-valerolactone) or poly(ε-caprolactone) or copolymers thereof, or poly(δ-valerolactone). In another embodiment, the A block comprises a copolymer of poly(δ-valerolactone) and glycolic acid.

In an embodiment of the disclosure, the B block comprises polyethylene glycol.

In another embodiment, the hydrophilic B block hydrophilic segments may also contain ionizable groups, if for example, B-A-B type copolymers are used.

In another embodiment of the disclosure, the block copolymer includes

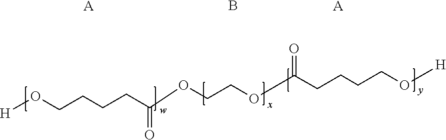

wherein the integers w, x and y represent the number of repeating units to obtain a block copolymer wherein the A block has a number average molecular weight between 500 and 30,000 and the B block has a number average molecular weight between 500 and 10,000.

In one embodiment, the block copolymers of the present disclosure possess water-solubility and gelling properties, such that the copolymers possess water solubility at temperatures below the gelling temperature and that there is rapid gelation under physiological conditions (for example, a temperature of about 37° C.). Accordingly, in one embodiment, when the copolymers are conjugated to, or contain therapeutic compounds, and administered to a patient, the rapid gelling minimizes the initial burst of the therapeutic compound, such as a cell or cytokines. In one embodiment, the temperature at which the block copolymers gel (or the reverse thermal gelation temperature) is controlled by the molecular weights, i.e. molar ratios, of the A block and the B block in the block copolymer.

In one embodiment of the disclosure, the hydrophobic A block comprises about 20% to 80% by weight of the copolymer, optionally 30% to 70%, or about 50%, and the hydrophilic B block makes up 80% to 20% by weight of the copolymer, optionally, 70% to 30%, or about 50%.

In another embodiment, the concentration at which the block copolymers of the present disclosure are soluble in aqueous solution, (e.g., water, buffer, etc.) below the gelling temperature is up to about 60% by weight, or optionally about 10% to about 40%.

In another embodiment, a block copolymer is provided that includes at least one A block and at least one B block, wherein the block copolymer has the formula:

A-B-A

wherein the A block comprises poly(δ-valerolactone) or poly(ε-caprolactone), or a co-polymer thereof, all of which are optionally substituted; wherein the B block comprises polyethylene glycol, which is optionally substituted; wherein the A block has a number average molecular weight between 500 and 10,000 and the B block has a number average molecular weight between 1,000 and 8,000; wherein the molecular weight ratio of A to B is between about 1.15 and about 1.25; wherein the optional substituents are selected from halo, OH, (C₁₋₆)-alkyl and fluoro-substituted (C₁₋₆)-alkyl; wherein the polymer is further functionalized with a vascular growth agent, and wherein the block copolymer forms a hydrogel at a temperature of above about 30° C.

Also provided is a temperature sensitive injectable hydrogel formulation for use in treating a vascular abnormality. The hydrogel formulation includes: a triblock polymer including blocks of biodegradable polymer having substantially equal number average molecular weights such that a honeycomb structure is formed above 30° C. with a pore size of about 1 μm, and a vascular growth agent conjugated to the polymer, wherein the formulation is injectable at ambient temperature, gels at body temperature, and substantially or completely degrades within 2 months. In some embodiments the pore size is between about 0.5 μm and about 10 μm, in some embodiments the pore size is between about 0.5 μm and about 5 μm, in some embodiments the pore size is between about 0.5 μm and about 2 μm, in some embodiments the pore size is between about 0.5 μm and about 1.5 μm, in some embodiments, the pore size is between about 1 μm and 5 μm, and in some embodiments the pore size is between about 1 μm and about 2 μm,. The term “pore size” means average pore size.

In another aspect, a method is provided for treating a vascular abnormality. The method includes administering any of the temperature sensitive hydrogel formulations described herein, including the hydrogel disclosed directly above, to a site of vascular abnormality, such that the vascular abnormality is treated.

### (III) Process of the Disclosure

The present disclosure also includes processes for the preparation of block copolymers including at least one A block and at least one B block, having the formula A-B, A-B-A or B-A-B. In one embodiment, a hydrophilic B block polymer, such as polyethylene glycol is used as a cationic macro-initiator for the polymerization reaction with the monomeric subunits of the A block polymer, in which the cationic macro-initiator begins the cationic polymerization with biodegradable monomeric units of the B block. Accordingly, the block copolymers of the disclosure comprise biodegradable linkages, which are hydrolyzed in vivo and excreted through the kidney. In one embodiment, the process of the disclosure using a hydrophilic B block polymer, such as polyethylene glycol, increases the processability of higher molecular weight B block polymers, such as polyethylene glycol.

The present disclosure also includes a process for the preparation of a block copolymer including at least one A block and at least one B block having the formula A-B, A-B-A, B-A-B, the process including reacting

an optionally substituted polyethylene glycol or polypropylene glycol including the B block; with, (ii) monomeric units of the A block, the monomeric units including δ-valerolactone, ε-caprolactone, lactide, an α-hydroxy acid, glycolic acid, an anhydride, an ester, an orthoester, an etherester, an esteramide, a carbonate, a cyanoacrylate, a urethane, an acrylate, or a mixture thereof, all of which are optionally substituted, wherein the optional substituents are selected from halo, OH, (C₁₋₅)-alkyl and fluoro-substituted (C₁₋₆)-alkyl; in the presence of an acid catalyst having a pKa of less than −12, and wherein the process is performed at a temperature between −10° C. and 35° C.

In another embodiment, the number average molecular weight of the A block is controlled by the molar ratio of the monomeric units of the A block to the B block during the process.

In another embodiment of the process, the A block has a number average molecular weight between 500 and 30,000 and the B block has a number average molecular weight between 500 and 10,000.

In another embodiment, the A block has a number average molecular weight between 500 and 10,000, or 1,000 and 5,000, or 1,000 and 3,000, or about 1,750. In another embodiment, the B block has a number average molecular weight of between 1,000 and 8,000, optionally between 1,500 and 8,000, or 1,500 and 5,000.

In another embodiment of the process, the block copolymer has the formula: A-B-A.

In another embodiment of the process, the monomeric units of the A block comprise δ-valerolactone, ε-caprolactone, lactide, an α-hydroxy acid, glycolide or a copolymer thereof, optionally δ-valerolactone or ε-caprolactone or a copolymer thereof, or δ-valerolactone. In another embodiment, the A block comprises a copolymer of poly(δ-valerolactone) and glycolic acid.

In an embodiment of the process, the B block comprises polyethylene glycol.

In a further embodiment of the process, the acid catalyst is a sulfonic acid, preferably trifluoromethanesulfonic acid or fluorosulfonic acid. In an embodiment, the sulfonic acid is trifluoromethanesulfonic acid. Sulfonic acid catalysts are not indicated for use in cationic ring-opening polymerization for monomers disclosed in the present disclosure, for example sulfonic acid catalyst are not used with δ-valerolactone cationic polymerization to prepare triblock copolymers of the present disclosure.

In another embodiment of the disclosure, there is also included a block copolymer including at least one A block and at least one B block, wherein the block copolymer has the formula: A-B; A-B-A; or B-A-B; in which the block copolymer is produced by the process of the disclosure. In another embodiment, the block copolymer produced by the process comprises

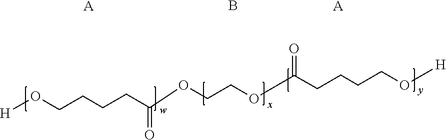

wherein the integers w, x and y represent the number of repeating units to obtain a block copolymer wherein the A block has a number average molecular weight between 500 and 30,000 and the B block has a number average molecular weight between 500 and 10,000.

In one embodiment, the mole ratio of B block to the monomeric units of the A block controls the lengths of the A blocks, and can provide a series of polymers with increasing A block contents and hydrophobicities.

In one embodiment, the process of the disclosure follows a scheme as shown in Scheme 1, in which for example, polyethylene glycol is the B block polymer, and δ-valerolactone is the monomeric unit forming the A block, to form a PVL-PEG-PVL triblock copolymer:

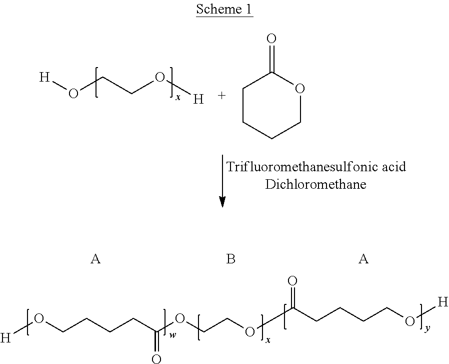

### (IV) Uses

The present disclosure also includes a pharmaceutical conjugate including a block copolymer as defined in the disclosure and a therapeutic compound. In another embodiment, the therapeutic compound is a biologic, optionally stem cell factor (SCF) or vascular endothelial growth factor (VEGF).

The present disclosure also includes a method for the treatment of cardiac abnormality and/or vascular abnormality in a patient in need thereof including administering a therapeutically effective amount of a pharmaceutical conjugate as defined in the disclosure to the site of the cardiovascular defect. In one embodiment, the cardiac abnormality is myocardial infarction and the vascular abnormality is a vascular aneurysm.

In one embodiment, the block copolymers are carboxy-derivatized to allow for the conjugation of a therapeutic compound, such as a biologic, such as VEGF or stem cell factors. For example, in one embodiment, the block copolymer is derivatized using a compound which adds a carboxyl group to the ends of the polymers, such as succinic anhydride, to obtain block copolymers with succinic acid groups at one or both ends of the polymer chain, which can be conjugated to cytokine such as VEGF. For example, in one embodiment, as shown in Scheme 2, the triblock copolymer as shown in Scheme 1 is further derivatized:

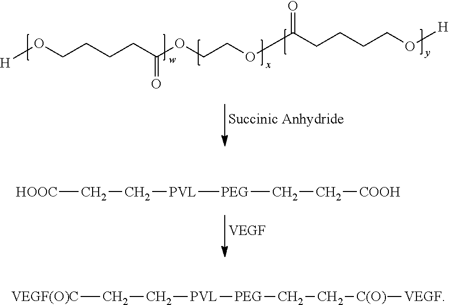

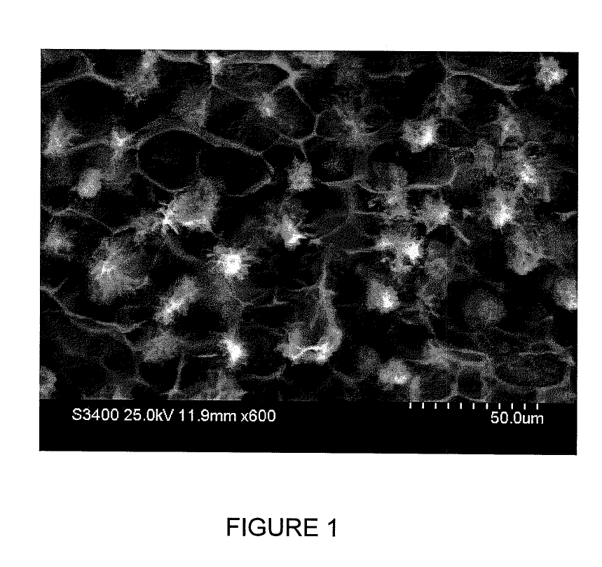

In one embodiment, the process used to mix the copolymers with a biologically active agent and/or other materials involves dissolving the block copolymers in an aqueous solution, followed by addition of the biologically active agent (in solution, suspension or powder such as VEGF and bone marrow cells), followed by thorough mixing to assure a homogeneous distribution of the biologically active agent throughout the copolymer. For example, FIG. 1 shows a photomicrograph of illustrating the distribution of rat bone marrow stromal cells in a block copolymer gelled matrix. FIG. 1 is a representative scanning electron micrograph illustrating that the hydrogel has a honey-comb structure with a pore size of about 1 μm. In another embodiment, the process involves dissolving the block copolymer in a biologically active agent-containing solution. In both embodiments, the process is conducted at a temperature lower than the gelation temperature of the copolymer and the material is implanted into the body as a solution which then gels into a depot in the body. The advantage of mixing an agent or material with the copolymers while in solution is both the uniform distribution of the agent or material in the formed hydrogel, as well as not being limited in the amount of agent or material that may be mixed or loaded based on diffusion or steric hindrance limitations that occurs with loading agents or materials into pre-formed hydrogels. In one embodiment, the biologically active agent will generally have a concentration in the range of 0 to 100 mg/mL or, if cells, a range of 100 to 10 million cells.

In another embodiment, buffers that may be used in the preparation of the biologically active agent-containing hydrogels are buffers which are well known by those of ordinary skill in the art and include sodium acetate, Tris, sodium phosphate, MOPS, PIPES, MES and potassium phosphate, in the range of 25 mM to 500 mM and in the pH range of 4.0 to 8.5.

In another embodiment, other excipients, e.g., various sugars (glucose, sucrose), salts (NaCl, ZnCl) or surfactants, are included in the biologically active agent-containing hydrogels of the present disclosure.

In one embodiment, proteins contemplated for use include but are not limited to interferon consensus (see, U.S. Pat. Nos. 5,372,808, 5,541,293 4,897,471, and 4,695,623 each of which are hereby incorporated by reference in their entirety), stem cell factor (PCT Publication Nos. 91/05795, 92/17505 and 95/17206, each of which are hereby incorporated by reference in their entirety) and rat VEGF. In addition, biologically active agents can also include fibroblast growth factors (FGF), insulin and Vascular endothelial growth factor (VEGF). The term proteins, as used herein, includes peptides, polypeptides, consensus molecules, analogs, derivatives or combinations thereof. In addition, in one embodiment, the block copolymers are useful for the treatment of damaged/diseased organs (or organ failure). In another embodiment, the block copolymers are useful for drug delivery in oncology via injection of the block copolymers (as conjugates and/or drug delivery devices) directly into a tumor mass or the use of the polymers in conjunction with photodynamic or temperature sensitive therapies into solid tumor masses.

The present disclosure also includes a hydrogel including a block copolymer as defined in the disclosure, transplanted cell or cells for transplantation, and a therapeutic compound. In another embodiment, the transplanted cells are autologous, homologus (allogenic) or xenogenic to the patient, and the compound is an immunosuppressant.

In one embodiment, the cells may be cells obtained or derived from a mammalian tissue. In another embodiment, the cells may include cardiomyocytes, smooth muscle cells, endothelial cells, bone marrow stem cells, stem cells in blood circulation, chondrocytes, chondroblasts, osteocytes and osteoblasts, periodontal cells, islet cells, or cells derived from skin and/or combinations thereof. In one embodiment, the cells are obtained from or derived from the living individual mammal, i.e. are autologous. In a preferred embodiment, the cells include Islet cells for the treatment of diabetes (e.g., type 1 diabetes mellitus). The cells may also be homologous, i.e. compatible with the tissue to which they are applied, or may be derived from multipotent or even pluripotent stem cells, for instance in the form of allogenic cells. In another embodiment the cells may be allogenic, from another similar individual, or xenogenic, i.e. derived from an organism other than the organism being treated. The allogenic cells could be differentiated cells, progenitor cells, or cells whether originated from multipotent (e.g., embryonic or combination of embryonic and adult specialist cell or cells, pluripotent stem cells (derived from umbilical cord blood, adult stem cells, etc.), engineered cells either by exchange, insertion or addition of genes from other cells or gene constructs, the use of transfer of the nucleus of differentiated cells into embryonic stem cells or multipotent stem cells, e.g., stem cells derived from umbilical blood cells.

In one embodiment, the immunosuppressant may be any compounds which can treat, prevent or reduce cell rejection. The immunosuppressant may be selected from the group consisting of PGE2, interleukins, cyclosporin, cyclophosphamide, FK506, rapamycin, corticosteroids, mycophenolate mofetil, leflunomide, deoxyspergualin, azathioprine, and OKT-3. Most preferably, the immunosuppressant is PGE2 or interleukin-10.

The present disclosure also includes a method for treating or preventing cell transplant rejection or prolonging the survival of transplanted autologous, homologus (allogenic) or xenogenic cells in a patient in need thereof, including administering a therapeutically effective amount of a compositions as defined in the disclosure.

In some embodiments, the compositions, materials and methods include those published in Wu, Jun, et al., “Infarct Stabilization and cardiac repair with a VEGF-conjugated, injectable hydrogel,” Biomaterials 32 (2011) 579-586, the entire disclosure of which is incorporated herein by this reference.

In another embodiment, the present disclosure is directed to a method for establishing tumor or cancer cells in a host. For example, this xenograft model may be capable of establishing tumors from primary tumors via injection of tumor cells into a host (e.g., immunodeficient mice). See U.S. Pat. No. 8,044,259, the entire contents of which is hereby incorporated by reference.

The method of establishing the cells may include preparing a mixture of a block copolymer of the present disclosure and the cells to be established; administering the mixture to a host; and growing the cells in the host, wherein the mixture forms a temperature sensitive hydrogel upon administration to the host.

The tumor or cancer cells may be from any known tumor or cancer cells, including fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, liver cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. The cells may also have a reduced, impaired or inherently low capacity for proliferation and the ability to give rise to new tumors.

The host may be any mammal known in the art for use in the transplant and proliferation of tumors, including nude mice, rats, etc.

The mixture may be administered to the host by any method known in the art. For example, the cells may be introduced to the host by injecting the cells in the mammary gland of the host.

The mixture may also comprises one or more therapeutic compounds known in the art to promote cell growth and/or treat, prevent or reduce cell rejection. The therapeutic compounds may be selected from growth factors (e.g., VEGF) and immunosuppressants (e.g., PGE2 and IL-10). These therapeutic compounds may be admixed in the mixture or conjugated to the block copolymer.

The following non-limiting examples are illustrative of the present invention:

### EXAMPLES

### Example 1

### Hydroxy-Terminated A-B-A Block Copolymer (polyvalerolactone)-(polyethylene glycol)-(polyvalerolactone) (PVL-PEG-PVL)

0.5 g polyethylene glycol (PEG) (0.33 mmol) and 1.2 g valerolactone (VL, 12 mmol) were dissolved in 5 mL dichloromethane. Trifluorimethanesulfonic acid catalyst, 61 μL (0.67 mmol) was added to the mixture at 0° C. The reaction was maintained for 3 hours and terminated by the addition of 0.2 g of NaHCO₃, and then the mixture was filtered. The copolymer was collected after precipitation in hexane and dried in the oven.

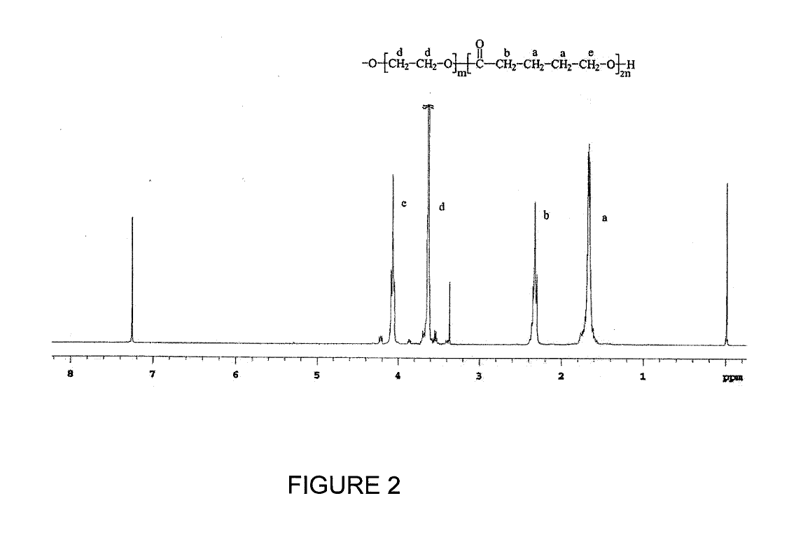

The molecular weight of the poly-VL (PVL) block was calculated from ¹H nuclear magnetic resonance, with the known molecular weight of the PEG precursor used as reference and CHCl₃ as the internal standard. The isolated polymer was dried at 40° C. under vacuum for 48 hours. The molecular weight of the block copolymer was determined by gel permeation chromatography (GPC) using polystyrene standards. The copolymer composition and relative block lengths were determined by ¹H-NMR (as shown in FIG. 2). The molecular weight of the PVL-PEG-PLV tri block copolymer was 1800-1500-1800, respectively. The PVL-PEG-PVL tri block copolymer was dissolved in 100 mM sodium phosphate, pH 7.4, and exhibited the thermo-reversible property (solution below room temperature and gel above room temperature).

### Example 2

### Hydroxy-Terminated A-B-A Block Copolymer (polyvalerolactone)-(polyethylene glycol)-(polyvalerolactone) (PVL-PEG-PVL)

This example describes synthesis of a hydroxy-terminated A-B-A (PVL-PEG-PVL), tri block copolymer by cationic polymerization method using a polyethylene glycol macro-initiator having a molecular weight of (Mn=5,000).

0.5 g polyethylene glycol (PEG) (0.132 mmol) and 1.2 g valerolactone (VL, 12 mmol) were dissolved in 5 mL dichloromethane. Trifluorimethanesulfonic acid catalyst, 24 μL (0.27 mmol) was added to the mixture at 0° C. The reaction was maintained for 3 hours and terminated by the addition of 0.1 g of NaHCO₃, and then the mixture was filtered. The copolymer was collected after precipitation in hexane and dried in the oven.

The molecular weight of the poly-VL (PVL) block was calculated from ¹H nuclear magnetic resonance, with the known molecular weight of the PEG precursor used as reference and CHCl₃ as the internal standard. The isolated polymer was dried at 40° C. under vacuum for 48 hours. The molecular weight of the block copolymer was determined by gel permeation chromatography (GPC) using polystyrene standards. The copolymer composition and relative block lengths were determined. The molecular weight of the PVL-PEG-PLV tri block copolymer was 6000-5000-6000, respectively. The PVL-PEG-PVL tri block copolymer dissolved either in 100 mM sodium phosphate, pH 7.4, exhibited the thermo-reversible property (solution below room temperature and gel above room temperature, or about 30° C.).

### Example 3

### Hydroxy-Terminated A-B-A Block Copolymer (polyvalerolactone)-(polyethylene glycol)-(polyvalerolactone) (PVL-PEG-PVL)

This example describes synthesis of a hydroxy-terminated A-B-A (PVL-PEG-PVL), tri block copolymer by cationic polymerization method using a polyethylene glycol macro-initiator having a molecular weight of (Mn=8,000).

0.5 g polyethylene glycol (PEG) (0.0625 mmol) and 1.2 g valerolactone (VL, 12 mmol) were dissolved in 5 mL dichloromethane. Trifluorimethanesulfonic acid catalyst, 16 μL (0.17 mmol) was added to the mixture at 0° C. The reaction was maintained for 3 hours and terminated by the addition of 0.2 g of NaHCO₃, and then the mixture was filtered. The copolymer was collected after precipitation in hexane and dried in the oven.

The molecular weight of the polyvalerolactone (PVL) block was calculated from ¹H nuclear magnetic resonance, with the known molecular weight of the PEG precursor used as reference and CHCl₃ as the internal standard. The molecular weight of the PVL-PEG-PLV tri block copolymer was 9600-8000-9600, respectively.

TABLE 1
Molar Ratio and Gelation Temperature for Tri Block Copolymers

| | A Block | B Block | A Block | Molar | Gelation |
|---|---|---|---|---|---|
| Example | mol. wt | mol. wt | mol. wt. | Ratio | Temp (° C.) |
| 1 | 1800 | 1500 | 1800 | 1.2x-1.0x-1.2x | 37 |
| 2 | 6000 | 5000 | 6000 | 1.2x-1.0x-1.2x | 32 |
| 3 | 9600 | 8000 | 9600 | 1.2x-1.0x-1.2x | 28 |
| 4 | 1500 | 1500 | 1500 | 1.0x-1.0x-1.0x | 42 |
| 5 | 1200 | 1000 | 1200 | 1.2x-1.0x-1.2x | 30 |

### Example 4

### Carboxy-Terminated A-B-A Block Copolymer (polyvalerolactone)-(polyethylene glycol)-(polyvalerolactone) (PVL-PEG-PVL)

This example describes modification of hydroxy-terminated PVL-PEG-PVL tri block copolymer to carboxylic acid-terminated PVL-PEG-PVL tri-block copolymer.

To a hydroxy-terminated PVL-PEG-PVL copolymer (1.0 grams) as described in Example 1, 10 ml of anhydrous 1,4-dioxane was added under continuous nitrogen purging. After complete dissolution of the polymer, 1.0 grams of succinic anhydride in 1,4-dioxane was added, followed by addition of 0.2 grams triethylamine and 0.1 grams of 4-dimethylaminopyridine. The reaction mixture was stirred at room temperature for 24 hours under nitrogen atmosphere. The conversion of terminal hydroxyl groups to carboxylic acid groups was followed by IR spectroscopy. After completion of the reaction the crude block polymer was isolated by precipitation using ether. The crude acid-terminated polymer was further purified by dissolving the polymer in methylene chloride (40 ml) and precipitating from ether. The isolated polymer was dried at 40° C. under vacuum for 48 hours.

The dried acid-terminated block copolymer (0.8 grams) was dissolved in 10 ml of 100 mM sodium phosphate buffer (pH 7.4), and filtered through 0.45 μm filter. The polymer solution was then placed in a dialysis membrane (2,000 Molecular Weight cut-off) and dialyzed against deionized water at 4° C. After dialysis, the polymer solution was lyophilized and the dried polymer was stored at −20° C. under a nitrogen environment.

The molecular weight of the tri block copolymer was determined by gel permeation chromatography (GPC) using polystyrene standards. The copolymer composition and relative block lengths were determined by ¹H-NMR.

### Example 5

### N-hydroxysuccinimide-Terminated A-B-A Block Copolymer (polyvalerolactone)-(polyethylene glycol)-(polyvalerolactone) (PVL-PEG-PVL)

The N-Hydroxysuccinimide-terminated block copolymer was synthesized by reacting the carboxy-terminated triblock copolymer with succinic anhydride.

Synthesis of NHS-terminated block copolymer: In a round-bottom flask equipped with a magnetic stir bar and a rubber septum, attached to a nitrogen line and a bubbler, the following materials were added: 0.5 g of dicarboxy-terminated block copolymer (0.128 mmol), 0.0396 g of N,N-dicyclohexylcarbodiimide (1.5× excess, 0.192 mmol), 0.0221 g of N-hydroxysuccinimide (1.5 excess, 0.192 mmol), and 5 mL of dichloromethane. The reaction was maintained for 24 h at room temperature. The reaction mixture was then filtered, and precipitated in cold diethyl ether. This reaction produced 0.31 g for a yield of 62%.

### Example 6

### VEGF-Conjugated with Carboxy-Terminated A-B-A Block Copolymer (polyvalerolactone)-(polyethylene glycol)-(polyvalerolactone) (PVL-PEG-PVL)

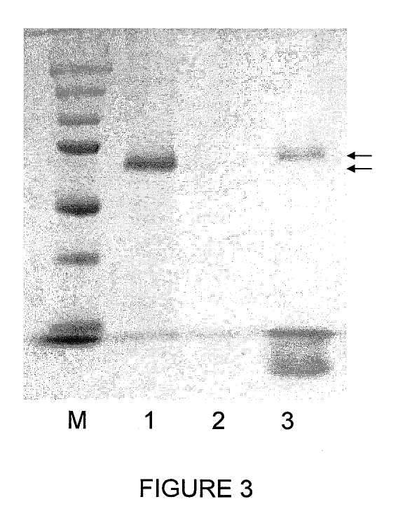

This example describes synthesis of a VEGF conjugate from a carboxylic acid-terminated PVL-PEG-PVL tri block copolymer. 10 mg NHS-PVL-PEG-PVL-NHS (from Example 5) was added to a solution of 100 ng VEGF in 0.5 mL phosphate buffered saline (PBS; pH=7.4, equiv. 200 ng/mL). The reaction was maintained for 24 h at room temperature. To remove the uncoupled VEGF, the reaction mixture was dialyzed against water/PBS buffer using Spectra/Por 2 dialysis membrane tubing with a molecular weight cut-off of 12-14 kDa for 48 h. The reaction product (VEGF conjugate) was analyzed with sodium dodecyl sulfate-polyacrylamide gel electrophoresis and the VEGF proteins were stained with Coomassie Brilliant Blue, as shown in FIG. 3. In FIG. 3, “M” refers to a protein ladder marker, Lane 1 refers to VEGF protein as a positive control, Lane 2 refers to the hydrogel alone as negative control and Lane 3 refers to the hydrogel conjugated with VEGF.

### Example 7

### pH Dependent Gelation/De-Gelation of A-B-A Block Copolymer (polyvalerolactone)-(polyethylene glycol)-(polyvalerolactone) (PVL-PEG-PVL)

### (a) Gelation

The following example demonstrates temperature dependent gelation of a PVL-PEG-PVL tri block copolymer solution.

The tri-block copolymer of PVL-PEG-PVL as described in Example 1 was dissolved in sodium phosphate buffers to obtain 20% (by weight) polymer solution with final pH in the range of 7.0-8.0. One milliliter polymer solution, formulated in different pH buffers, was placed in a glass vial at 37° C. and the gelation was monitored visually as a function of time.

### (b) De-Gelation

The following example demonstrates temperature dependent de-gelation (gel to solution) of the PVL-PEG-PVL hydrogel.

The above gel (from Example 7b) was heated to 60° C. at which point it became a liquid solution. Then, it was cooled down to room temperature and became a solution again.

### Example 8

### Temperature-Sensitive A-B-A Block Copolymer (polyvalerolactone)-(polyethylene glycol)-(polyvalerolactone) (PVL-PEG-PVL)

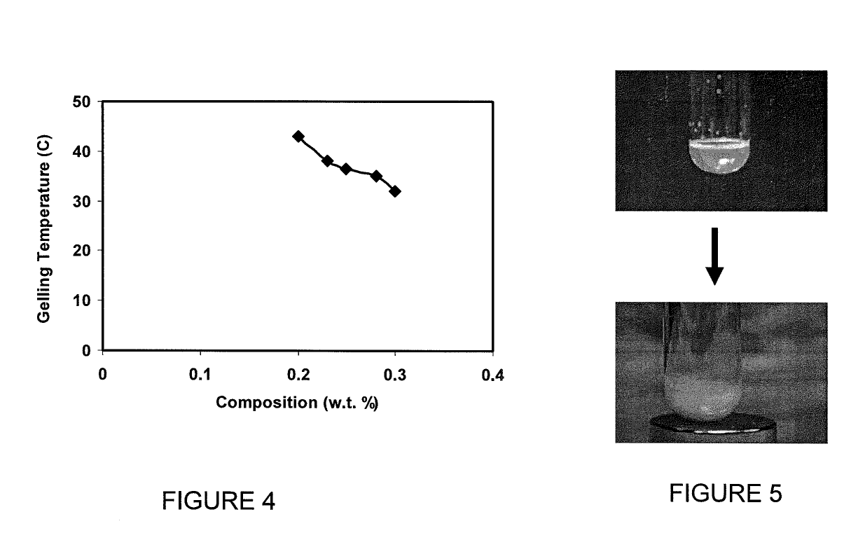

The temperature-sensitive hydrogel was prepared as follows: 0.2 g PVL-PEG-PVL was added to 0.8 mL PBS. The mixture was heated to 60° C. and stirred until the polymer was completely dissolved, and then cooled to 10° C. A clear polymer solution formed. The gelling temperature was determined by increasing the temperature by 5° C. per min until a gel formed. A VEGF-conjugated hydrogel (HG−VEGF) was prepared by adding VEGF conjugates to PVL-PEG-PVL solution at 10° C. The results are summarized in FIGS. 4 and 5, in which in FIG. 4 the graph illustrates the temperature dependent gelation at a pH above 7.4. FIG. 5 illustrates the gelling process in which a solution of the block copolymer is heated from room temperature to about 37° C. (10 minutes) to form a hydrogel.

### Example 9

### Degradation of A-B-A Block Copolymer (polyvalerolactone)-(polyethylene glycol)-(polyvalerolactone) (PVL-PEG-PVL)

This example describes the hydrogel degradation in vitro and in vivo. PVL-PEG-PVL (10, 20 or 30 μL) was added onto a cell culture dish formed a gel at 37° C. The nodule diameter did not change for 4 weeks, indicating the gel was stable in vitro. The hydrogel injected subcutaneously (10, 20, or 30 μL) into a rat, which immediately absorbed water and the nodule size initially increased at all 3 tested concentrations. However, nodule size decreased beyond 7 days after implantation and the nodules were completely degraded after 42 days indicating biodegradation of the hydrogel copolymer.

### Example 10

### Comparison Studies of Hydrogel (PVL-PEG-PVL) and VEGF Conjugate of Hydrogel

Myocardial infarction were created from a total of 44 Sprague Dawley rats (body weight=200-250 g) and were used for the studies. 100 uL of PBS, HG, HG mixed with VEGF (40 ng/rat) (HG+VEGF), or HG−VEGF (40 ng VEGF/rat) was injected into 4 sites around the infarct area with a 28-gauge insulin syringe (25 uL/injection), and the incision was closed. All animals received post-operative care. Function was evaluated at 35 days after injection with a pressure volume catheter. The heart function was improved.

After the pressure/volume analysis was complete, hearts were rapidly excised and fixed in 10% formaldehyde. Morphometry analysis were performed. It was determined that hydrogel prevented ventricular dilation.

### Discussion

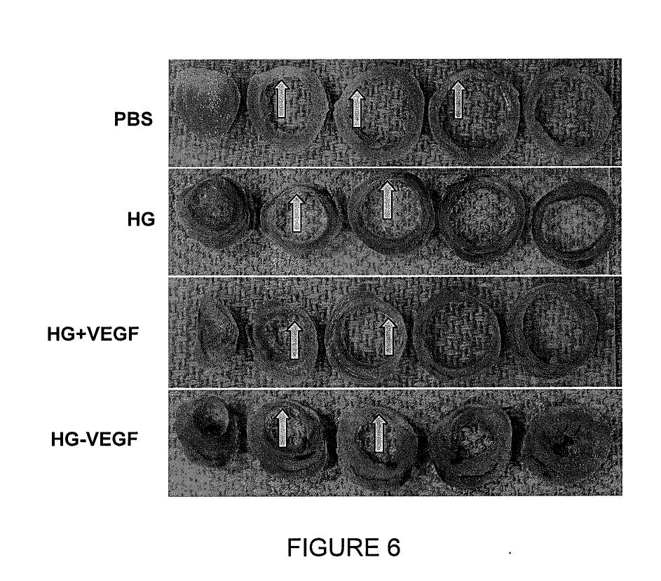

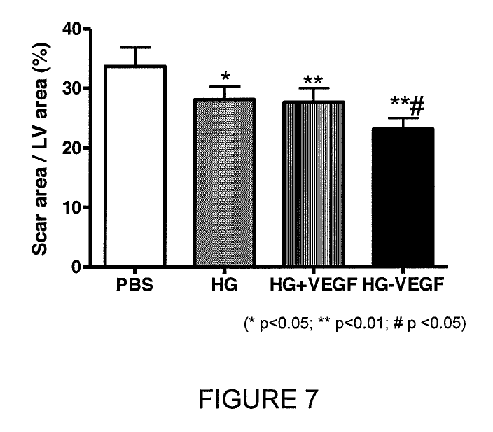

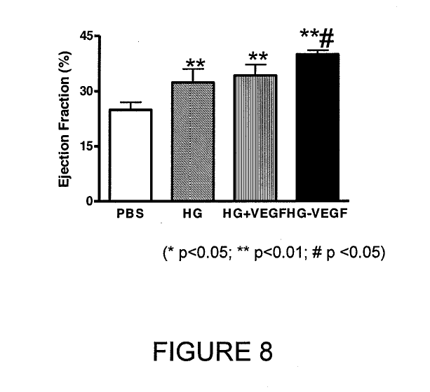

As shown in FIG. 6, representative heart slices obtained at 35 days after myocardial infarction with injection of PBS, HG (hydrogel copolymer), HG+VGF (mixture of hydrogel and VEGF) and HG−VEGF (conjugate of VEGF and hydrogel), wherein the arrows indicate the location of the infarct in individual slices, and illustrates that HG, HG+VEGF or HG−VEGF helps to prevent scar expansion. As shown in FIG. 7, the left ventricular scar area after myocardial infarction is lower when treated with the HG, HG+VEGF or HG−VEGF. Finally, FIG. 8 illustrates that the ejection fraction of the heart was greater when treated with HG, HG+VEGF or HG−VEGF.

It was determined that the block copolymer hydrogel of the present disclosure provides a temporary scaffold to attenuate adverse cardiac remodeling and helps to prevent scar expansion. The block copolymer also provides a platform for the sustained release of a therapeutic compound (VEGF). In this Example, VEGF further attenuated adverse cardiac remodeling, stimulates angiogenesis and prevents heart failure. The sustained release of VEGF stimulates new blood vessel formation and when VEGF is conjugated with block copolymer, they act synergistically to impede scar expansion, maintain LV structure and preserve LV function.

### Example 11

### Experimental Procedure of PVL-PEG-PVL Gel as a Carrier for Prostaglandin E2 (PGE2) Delivery In Vivo of Rat Model

PGE2 is an immunosuppressant which may inhibit T cell activation in vitro and may prevent or inhibit cell rejection after cell transplantation. PGE2 and bone marrow cells were fixed in a PVL-PEG-PVL hydrogel and tested in an in vivo rat model. The solubility of PGE2 is about 5 mg/mL (which is the Critical Micelle Concentration, CMC) at a pH above 6. Since PGE2 has very short half life under physiological condition, it is a challenge to achieve sustained concentration of PGE2 in vivo to prevent rejection and prolong survival of implanted cells. Since PVL-PEG-PVL hydrogel is temperature sensitive and biodegradable biomaterial, we proposed PGE2 delivery using our PVL-PEG-PVL hydrogel as a carrier.

Myocardial infarction: We used Sprague Dawley rats (body weight=200-250 g). All experiments were performed in accordance with the principles of laboratory animal care formulated by the guide for the care and use of laboratory animals by the Institute of Laboratory Animal Resources (Commission on Life Sciences, National Research Council). All animal procedures were approved by the University Health Network Animal Care Committee. Detailed surgical procedures for MI (coronary artery ligation) were as we previously described (Kan C D, Li S H, Weisel R D, Zhang S, Li R K. Recipient age determines the cardiac functional improvement achieved by skeletal myoblast transplantation. J Am Coll Cardiol 2007; 50:1086-92.). Cardiac function was evaluated using echocardiography at day 35 after myocardial infarction.

Hydrogel preparation: The triblock copolymer of PVL-PEG-PVL (200 mg) from example 1 was dissolved in phosphate buffered saline (800 μL). At room temperature, 50 ng of PGE2 (5 μL of stock solution containing 10 μg/mL in ethanol of PGE2) were mixed in 50 μL of polymer solution and three million of bone marrow cells homogeneously before injecting to the rat heart using a 28-gauge insulin syringe.

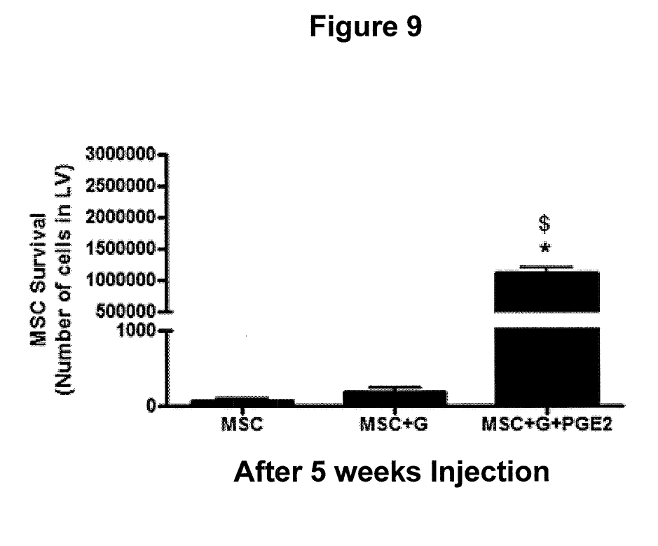

Hydrogel injection: Under general anesthesia with ventilation, the heart was exposed through a thoracotomy. Bone marrow cells, 50 μL of the hydrogel (example 1) mixed with three million of bone marrow cells, or hydrogel mixed with PGE2 (50 ng/rat) and three million of bone marrow cells were injected into 4 sites around the infarct with a 28-gauge insulin syringe (12.5 μL/injection), and the incision was closed. All animals received post-operative care. The survival rate of implanted cells was measured after injection for five weeks. FIG. 9 illustrates that at five weeks after cell injection, the survival rate of implanted cells is significant higher in cell+GPE2 group compared with cell alone or cells mixed with hydrogel group.

We claim:

1. A block copolymer comprising at least one A block and at least one B block, wherein the block copolymer has the formula:
A-B;
A-B-A; or
B-A-B;
wherein the A block comprises poly(δ-valerolactone), poly(ε-caprolactone), poly(lactide), poly(α-hydroxy acid), poly(glycolide), polyanhydride, polyester, polyorthoester, polyetherester, polyesteramide, polycarbonate, polycyanoacrylate, polyurethane, polyacrylate, or a co-polymer thereof, all of which are optionally substituted;
wherein the B block comprises polyethylene glycol or polypropylene glycol, both of which are optionally substituted;
wherein the A block has a number average molecular weight between 500 and 30,000 and the B block has a number average molecular weight between 500 and 10,000;
wherein the optional substituents are selected from halo, OH, (C₁₋₆)-alkyl and fluoro-substituted (C₁₋₆)-alkyl; and
wherein the block copolymer forms a hydrogel at a temperature of above about 30° C.

2. The block copolymer of claim 1, wherein the A block has a number average molecular weight between 500 and 10,000.

3. The block copolymer of claim 1, wherein the B block has a number average molecular weight of between 1,000 and 8,000.

4. The block copolymer of claim 1, wherein the block copolymer has the formula:
A-B-A.

5. The block copolymer of claim 1, wherein the A block is a poly(δ-valerolactone), poly(ε-caprolactone), poly(lactide), poly(α-hydroxy acid), poly(glycolide) or a copolymer thereof.

6. The block copolymer of claim 1, wherein the A block comprises poly(δ-valerolactone) or poly(ε-caprolactone).

7. The block copolymer of claim 1, wherein the A block comprises poly(δ-valerolactone).

8. The block copolymer of claim 1, wherein the B block comprises polyethylene glycol.

9. The block copolymer of claim 1, wherein the block copolymer comprises 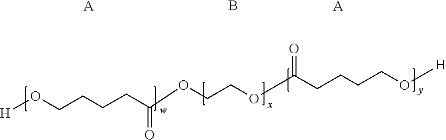
wherein the integers w, x and y represent the number of repeating units to obtain a block copolymer wherein the A block has a number average molecular weight between 500 and 30,000 and the B block has a number average molecular weight between 500 and 10,000.

10. The block copolymer of claim 1, wherein the molecular weight ratio of A to B is between about 1.05 and about 1.35.

11. The block copolymer of claim 1, wherein the molecular weight ratio of A to B is between about 1.15 and about 1.25.

12. The block copolymer of claim 1, wherein the molecular weight ratio of A to B is about 1.2.

13. The block copolymer of claim 1, wherein the block copolymer has the formula A-B-A, wherein the A block has a number average molecular weight of about 1200 and the B block has a number average molecular weight of about 1000.

14. The block copolymer of claim 1, wherein the block copolymer has the formula A-B-A, wherein the A block has a number average molecular weight of about 1800 and the B block has a number average molecular weight of about 1500.

15. The block copolymer of claim 1, wherein the block copolymer has the formula A-B-A, wherein the A block has a number average molecular weight of about 6000 and the B block has a number average molecular weight of about 5000.

16. The block copolymer of claim 1, wherein the block copolymer has the formula A-B-A, wherein the A block has a number average molecular weight of about 9600 and the B block has a number average molecular weight of about 8000.

17. A process for the preparation of a block copolymer comprising at least one A block and at least one B block having the formula A-B, A-B-A, or B-A-B, the process comprising reacting
(i) polyethylene glycol or polypropylene glycol comprising the B block, both of which are optionally substituted;
with,
(ii) monomeric units of the A block, the monomeric units comprising δ-valerolactone, ε-caprolactone, lactide, an α-hydroxy acid, glycolic acid, an anhydride, an ester, an orthoester, an etherester, an esteramide, a carbonate, a cyanoacrylate, a urethane, an acrylate, or a mixture thereof, all of which are optionally substituted, wherein the optional substituents are selected from halo, OH, (C₁₋₆)-alkyl and fluoro-substituted (C₁₋₆)-alkyl;
in the presence of an acid catalyst having a pKa of less than −12, and wherein the process is optionally performed at a temperature between −10° C. and 35° C.

18. The process of claim 17, wherein the number average molecular weight of the A block is controlled by the molar ratio of the monomeric units of the A block to the B block.

19. The process of claim 17, wherein the A block has a number average molecular weight between 500 and 30,000 and the B block has a number average molecular weight between 500 and 10,000.

20. The process of claim 17, wherein the acid catalyst is a sulfonic acid.

21. The process of claim 17, wherein the acid catalyst is trifluoromethanesulfonic acid or fluorosulfonic acid.

22. A block copolymer comprising at least one A block and at least one B block, wherein the block copolymer has the formula:
A-B;
A-B-A; or
B-A-B;
produced by the process as defined in claim 17.

23. A pharmaceutical composition comprising a block copolymer as defined in claim 1 and a therapeutic compound, wherein the therapeutic compound is conjugated to the copolymer.

24. The pharmaceutical composition of claim 23, wherein the therapeutic compound is a biologic.

25. The pharmaceutical composition of claim 23, wherein the biologic is stem cell factor (SCF) or vascular endothelial growth factor (VEGF).

26. A method for the treatment of cardiac abnormality and/or vascular abnormality in a patient in need thereof comprising administering a therapeutically effective amount of a pharmaceutical composition as defined in claim 23 to the site of the cardiovascular defect.

27. The method of claim 26 wherein the cardiac abnormality is myocardial infarction.

28. The method of claim 26 wherein the vascular abnormality is a vascular aneurysm.

29. A pharmaceutical composition comprising a block copolymer as defined in claim 1, a therapeutic compound and transplant cells.

30. The pharmaceutical composition of claim 29, wherein the therapeutic compound is an immunosuppressant.

31. The pharmaceutical composition of claim 29, wherein the therapeutic compound is selected from the group consisting of PGE2, interleukins, cyclosporin, cyclophosphamide, FK506, rapamycin, corticosteroids, mycophenolate mofetil, leflunomide, deoxyspergualin, azathioprine, and OKT-3.

32. A method for treating or preventing cell transplant rejection in a patient in need thereof, comprising administering a therapeutically effective amount of a pharmaceutical composition as defined in claim 29.

33. The method of claim 32 wherein the therapeutically effective amount of the pharmaceutical composition is an amount effective to inhibit a T-cell mediated immune response in the patient to the transplanted cells.

34. The method of claim 32, wherein the cell transplant is autologous, homologus (allogenic) or xenogenic to the patient.

35. The method of claim 32, wherein the cell transplant comprises bone marrow cells.

36. A block copolymer comprising at least one A block and at least one B block, wherein the block copolymer has the formula:
A-B-A
wherein the A block comprises poly(δ-valerolactone) or poly(ε-caprolactone), or a co-polymer thereof, all of which are optionally substituted;
wherein the B block comprises polyethylene glycol, which is optionally substituted;
wherein the A block has a number average molecular weight between 500 and 10,000 and the B block has a number average molecular weight between 1,000 and 8,000;
wherein the molecular weight ratio of A to B is between about 1.15 and about 1.25;
wherein the optional substituents are selected from halo, OH, (C₁₋₅)-alkyl and fluoro-substituted (C₁₋₆)-alkyl;
wherein the polymer is further functionalized with a vascular growth agent, and
wherein the block copolymer forms a hydrogel at a temperature of above about 30° C.

37. A temperature sensitive injectable hydrogel formulation for use in treating a vascular abnormality, the hydrogel formulation comprising:
a triblock polymer comprising blocks of biodegradable polymer having substantially equal number average molecular weights such that a honeycomb structure is formed above 30° C. with a pore size of about 1 μm,
and a vascular growth agent conjugated to the polymer,
wherein the formulation is injectable at ambient temperature, gels at body temperature, and substantially or completely degrades within 2 months.

38. A method for treating a vascular abnormality comprising:
administering the temperature sensitive hydrogel formulation of claim 37 to a site of vascular abnormality, such that the vascular abnormality is treated.

39. A method for establishing tumor or cancer cells in a host comprising:
preparing a mixture of a block copolymer of claim 1 and the cells;
administering the mixture to the host; and
growing the cells in the host;
wherein the mixture forms a temperature sensitive hydrogel upon administration to the host.

40. The method of claim 39, wherein the mixture further comprises a therapeutic compound.

41. The method of claim 39, wherein the mixture further comprises a growth factor or an immunosuppressant.
